(19) United States
(12) Patent Application Publication
(10) Pub. No.: US 2024/0032796 A1
(43) Pub. Date: Feb. 1, 2024

(54) Compact all free-space line-field swept source OCT system

(71) Applicant: KineoLabs, Billerica, MA (US)

(72) Inventor: Walid A. Atia, Jamaica Plain, MA (US)

(21) Appl. No.: 18/359,337

(22) Filed: Jul. 26, 2023

Related U.S. Application Data
(60) Provisional application No. 63/393,594, filed on Jul. 29, 2022.

(51) Int. Cl. A61B 5/00; G01B 9/02091; G01B 9/02004; H01S 3/067; H01S 3/0941

(52) U.S. Cl. CPC A61B 5/0066; G01B 9/02091; G01B 9/02004; H01S 3/06783; H01S 3/0941

(57) ABSTRACT

A compact possibly all free-space line-field swept source OCT system with a tunable cat's-eye laser.

### RELATED APPLICATIONS

This application claims the benefit under 35 USC 119(e) of U.S. Provisional Application No. 63/393,594, filed on Jul. 29, 2022, which is incorporated herein by reference in its entirety.

### BACKGROUND OF THE INVENTION

Optical coherence tomography (OCT) is a cross-sectional, non-invasive imaging modality that is used in many areas of medical imaging to generate high-resolution cross-sectional images of various parts of the body. While it is primarily associated with ophthalmology, where it is used to image the retina and diagnose conditions like macular degeneration and glaucoma, it can also be used to image choroid and anterior segment. Functional imaging of the blood velocity and vessel microvasculature is also possible. In addition, OCT has also been adopted in or at least proposed in other medical domains. OCT is used in interventional cardiology to visualize coronary arteries and help with the placement of stents, among other procedures. It provides more detailed images than intravascular ultrasound (IVUS), allowing for better identification of plaque, thrombi, or malapposed stents. OCT has also been used to image the skin to assist in diagnosing and treating a variety of skin diseases. It can be used to detect changes in skin morphology associated with conditions like skin cancer, psoriasis, and dermatitis. It can further be used to image the gastrointestinal tract, helping to detect and diagnose conditions such as Barrett's esophagus, gastric cancer, and other abnormalities. Some have used OCT to visualize the respiratory tract, allowing for detailed imaging of airway structures and assisting in the diagnosis of conditions like asthma or chronic obstructive pulmonary disease (COPD). In otolaryngology, OCT can be used to image the vocal cords, middle ear, and other structures, helping with the diagnosis and treatment of conditions affecting these areas. OCT could also be used to visualize bladder tissue for diagnosis and management of bladder cancers.

Various architectures exist for OCT. Fourier-domain OCT (FD-OCT) has recently attracted more attention because of its high sensitivity and imaging speed compared to time-domain OCT (TD-OCT), which uses an optical delay line for mechanical depth scanning with a relatively slow imaging speed. The spectral information discrimination in FD-OCT is accomplished either by using a dispersive spectrometer in the detection arm (spectral domain or SD-OCT) or rapidly scanning a swept laser source (swept-source OCT or SS-OCT).

Compared to spectrometer-based SD-OCT, SS-OCT has several advantages, including its robustness to motion artifacts and fringe washout, lower sensitivity roll-off and higher detection efficiency.

Many different approaches have been investigated to develop high-speed swept sources for SS-OCT. One approach employs a semiconductor optical amplifier (SOA) based ring laser design (see for example Yun et al “High-speed optical frequency-domain imaging” Opt. Express 11:2953 2003 and Huber et al “Buffered Fourier domain mode locking: unidirectional swept laser sources for optical coherence tomography imaging at 370,000 lines/s,” Opt. Express 13, 3513 2005). Short cavity lasers (see for example Kuznetsov et al “Compact Ultrafast Reflective Fabry-Perot Tunable Lasers For OCT Imaging Applications,” Proc. SPIE 7554:75541F 2010) are another example. SOA based ring laser designs have been practically limited to positive wavelength sweeps (increasing wavelength) because of the significant power loss that occurred in negative tuning. This has been attributed to four-wave mixing (FWM) in SOAs causing a negative frequency shift in intracavity light as it propagates through the SOA (Bilenca et al “Numerical study of wavelength-swept semiconductor ring lasers: the role of refractive-index nonlinearities in semiconductor optical amplifiers and implications for biomedical imaging applications,” Opt. Lett. 31: 760-762 2006).

A commercially available short cavity laser (Axsun Technologies Billerica, MA) in excess of 100 kHz has been reported (see for example Kuznetsov et al “Compact Ultrafast Reflective Fabry-Perot Tunable Lasers for OCT Imaging Applications,” Proc. SPIE 7554: 75541F 2010). Short cavity lasers enable a significant increase in sweep speeds over conventional swept laser technology because the time needed to build up lasing from spontaneous emission noise to saturate the gain medium is greatly shortened (R. Huber et al “Buffered Fourier domain mode locking: unidirectional swept laser sources for optical coherence tomography imaging at 370,000 lines/s,” Opt. Express 13: 3513 2005). However, the effective duty cycle of the bidirectional sweeping short cavity laser was limited to less than 50% because of the FWM effects mentioned above. The effective repetition rate of the laser is thus limited.

More recently, tunable vertical cavity surface emitting lasers (VCSELs) have been offered by Thorlabs and Axsun Technologies. The short cavities implicit in this technology enables even higher speed sweeping.

Other methods have also been proposed to increase the effective repetition rates of SS-OCT systems including sweep buffering with a delay line, and multiplexing of multiple sources, thereby increasing the duty cycle of the laser. The method used to multiplex these sweeps together may include components that introduce orthogonal polarizations to the sweeps originating from different optical paths. Combining diverse polarizations at a polarization beamsplitter is a very light efficient way of transmitting the light to a single beam path.

Goldberg et al. demonstrated a ping-pong laser configuration for high-speed SS-OCT system that achieves a doubling of the effective A-line rate by interleaving sweeps of orthogonal polarization in the same cavity (see Goldberg et al “200 kHz A-line rate swept-source optical coherence tomography with a novel laser configuration” Proceedings of SPIE v.7889 paper 55 2011).

Potsaid et al. demonstrated another method to double the effective repetition rate of a swept source laser by buffering and multiplexing the sweep of a single laser source (see Potsaid et al “Ultrahigh speed 1050 nm swept source/Fourier domain OCT retinal and anterior segment imaging at 100,000 to 400,000 axial scans per second” Opt. Express 18: 20029-20048 2010). However, the long fiber spool will cause a significant birefringence to the laser output.

At the same time, other architectures exist for SS-OCT that reduce the performance requirements for the swept laser source, however. Fechtig, et al. in an article entitled Line-Field parallel swept source MHz OCT for structural and functional retinal imaging, Biomedical Optics Express 716, vol. 6, no. 3, (2015) describes a system that achieves 1 MHz equivalent A-scan rates by combining a lower sweep rate laser with a linear or line-scan sensor.

More recently, a SS-OCT architecture has been developed and disclosed in U.S. patent application Ser. No. 18/184,015, filed on Mar. 15, 2023 by Atia, et al, entitled Cat's-eye swept source laser for OCT and spectroscopy, which is incorporated herein by this reference in its entirety (hereinafter Atia). This is a line-field OCT system that employs a cat's-eye swept source laser.

### SUMMARY OF THE INVENTION

The present invention concerns a potentially compact line-field SS-OCT system. It can be an entirely free-space system in that no optical fiber is required. Thus, the system can also be very compact and completely integrated on a single bench.

In general, according to one aspect, the invention features an integrated line-field swept source OCT system comprising a base, a swept laser on the base, a beamsplitter on the base for dividing the beam from the swept laser between a reference arm and a sample arm, and a line-field sensor for detecting light from the reference arm and the sample arm.

In embodiments, a cylindrical lens, spherical lens, and/or an achromat lens is mounted to the base for conditioning the light from the swept laser to the beamsplitter.

A bracket can be used for mounting the line-field sensor to the base.

Preferably a line generating lens is used to help form a line from the beam from the laser. This line generating lens forms a less Gaussian and more of a flat-top power distribution of the light from the laser, and might be a Powell lens.

The base is usually generally t-shaped with the swept laser in the bottom and the reference arm and sample arm at the top. A translation stage is currently used to change the length of the reference arm.

In general, according to one aspect, the invention features an integrated line-field swept source OCT system comprising a base, a swept laser on the base, a beamsplitter on the base for dividing the beam from the swept laser between a reference arm and a sample arm, a line-field sensor for detecting light from the reference arm and the sample arm, and a line generating lens for forming a line from the beam from the laser.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIGS. 1 and 2 are perspective views of a line field swept source optical coherence tomography system according to the present invention.

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Also, all conjunctions used are to be understood in the most inclusive sense possible. Thus, the word “or” should be understood as having the definition of a logical “or” rather than that of a logical “exclusive or” unless the context clearly necessitates otherwise. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

It will be understood that although terms such as “first” and “second” are used herein to describe various elements, these elements should not be limited by these terms. These terms are only used to distinguish one element from another element. Thus, an element discussed below could be termed a second element, and similarly, a second element may be termed a first element without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

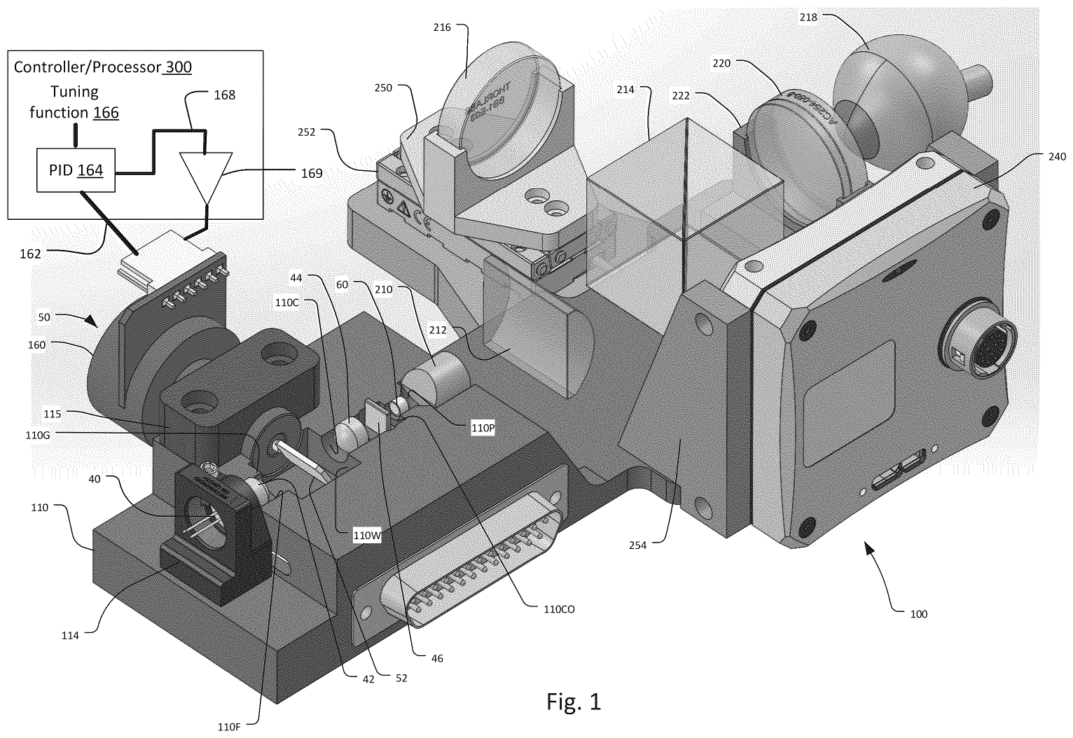

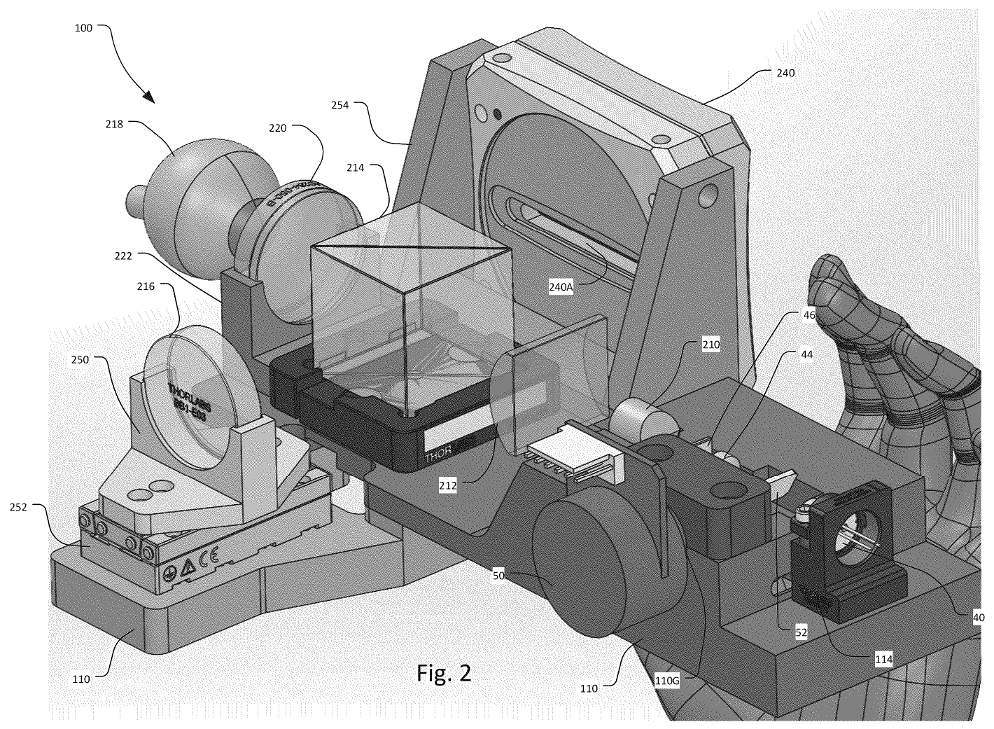

FIGS. 1 and 2 show a line-field free-space swept-source optical coherence tomography system (SS-OCT) 100, which has been constructed according to the principles of the present invention.

The system's swept source laser preferably employs a cats-eye architecture as described in Atia. The laser's amplification is provided by a GaAlAs gain chip in one example. The gain chip amplifies light in the wavelength range of about 800 to 900 nanometers. Preferably, the gain chip is an edge emitting, single angled facet device. Preferably its center wavelength is around 840 nanometers, which is useful for applications such as ophthalmic imaging. Another advantage of this wavelength range is that it can be detected with silicon, e.g., CMOS or CCD, imagers.

In the preferred embodiment, the gain chip is mounted in a TO-can type hermetic package 40. This protects the chip from dust and the ambient environment including moisture. In some examples, the TO-can package has an integrated or a separate thermoelectric cooler. In the preferred embodiment, the gain chip is operated coolerless with no thermoelectric cooler.

The free space beam from the package is diverging in both axes (x, y). It is collimated by a collimating lens 42. The resulting collimated beam is received by a cat's eye focusing lens 44, which focuses the light onto a cat's eye mirror/output coupler 46. This defines the other end of the laser cavity, extending between the mirror/output coupler and the back/reflective facet of the gain chip in the TO-can 40 and forms a cats-eye laser cavity.

The collimated light between the collimating lens and the cat's eye focusing lens passes through a bandpass filter 52. This is a thin film interference filter that provides a pass band of approximately 0.3 nanometers (nm). More generally, it is usually between 0.1 and 2 nanometers. Even more generally, it is between 0.05 nm to 5 nm filter linewidth. Note that the linewidths are measured at full width, half max (FWHM).

The bandpass filter 52 is held on an arm of a galvanometer 50 or other angular actuator. This allows for tilting of the bandpass filter in the collimated beam to thereby tilt tune the filter and thus change the passband to scan or sweep the wavelength of the swept laser.

Tuning speed specifications for the galvanometer generally range from 0.1 Hz to For the higher speeds, a 25 kHz resonant galvo can be used with bi-directional tuning, but higher and lower speeds can be used. Wavelength tuning speed is usually given in nm/sec, so for a typical 100 Hz tuning speed ideal for retinal imaging applications where a line-speed camera at 100 kHz will give 1000 sampled bandwidth points and 70 nm tuning range, this would give 70 nm/10 msec=7000 nm/sec. In general, the tuning speed should be between 3000 nm/sec 70000 nm/sec.

Tuning range specifications: For retinal or industrial imaging with low-cost CMOS cameras, 840 nm center wavelength is an ideal water window, and a minimum of 30 nm tuning range is possible but 70 nm or more of tuning is preferred for good resolution of <8 micrometers in air. In general, the tuning range is typically between 30 nm and 100 nm.

The galvanometer 50 is preferably operated as a servomechanism angle actuator. In the illustrated embodiment, the galvanometer 50 is a servo-controlled galvanometer. An encoder 160 in galvanometer's base produces an angle signal 162 indicating the angle of the galvanometer, and thus the filter 52, to the collimated beam. Preferably, the encoder is an optical encoder and is often analog.

A controller/processor 300 receives the angle signal 162 at a PID (proportional—integral—derivative) controller 164. The PID controller 164 compares the angle signal 162 to a specified tuning function 166. Often this tuning function is sawtooth or triangular waveform that is stored algorithmically or in a look up table in the controller/processor 300. It is defined to linearize the frequency versus time tuning of the laser. This yields feedback control system that corrects for any error in the position. The desired position dictated by the tuning function 166 is compared with the actual position of the galvanometer 50 to produce an error signal 168, which is then fed back to the galvanometer motor via an amplifier 169 to adjust the current and bring the filter 52 to the desired position.

The size of the collimated beam is important for many applications. As a general rule, a smaller beam results in higher divergence resulting in a larger cone half angle (CHA). This reduces the minimum line width over angle for a tunable filter. In the current embodiment, the collimated beam at the tunable filter 52 is not less than 1 millimeter (mm) and is preferably greater than 2 mm for retinal OCT application. In general, the CHA should be less than 0.04×0.02 degrees and preferably about 0.02×0.01 degrees or less.

The light from the gain chip is polarized. In the common architectures, the polarization is horizontal or parallel to the epitaxial layers of the edge-emitting gain chip. In the preferred configuration, the filter is oriented to receive the S polarization in order to maintain narrow line width of the filter as it is tilt tuned. On the other hand, the P polarization broadens drastically at large tilt angles. S polarization has higher loss at larger tilt angles than P. So, the filter design needs to address these issues by providing a low enough loss across the tuning band for S.

In general, the present cat's-eye laser configuration provides a number of advantages. It provides low loss, low tolerance, repeatable stable operation since lower angle wavelength change over grating-based lasers.

The mirror/output coupler 46 will typically reflect about 80% of the light back into the laser's cavity and transmits about 20% of light. Often, the transmitted light is collimated with the help of an output lens. More generally, the mirror/output coupler can reflect from 10% to 99% of light (transmitting 90% to 1%, respectively), depending on the output power and laser cavity loss desired. Higher reflectivity results in lower loss cavities and thus wider laser tuning range where gain exceeds loss, but results in lower output power.

In some embodiments, an iris or mask is added typically after the output coupler to clip the beam edge. This reduces power fluctuations as the beam wanders due to refraction in the tilting bandpass filter.

In the illustrated example, the fast axis of the chip is oriented horizontally in the figure, with the epitaxial layers of the chip being oriented vertically. Thus the beam transmitted through the mirror/output coupler 46 is elliptical with the long axis of the beam being vertical. This diverging elliptical beam is collimated by collimating output lens 60. Generally, the elliptical beam is between 1-4 millimeter wide in the long axis and about 0.5-2 millimeters in the short axis.

The elliptical beam is received by a line generating lens 210 such as a Powell lens, in one example. This generates a beam that has a less Gaussian than would be generated by a cylindrical lens. Instead, the line generating lens 210 produces a more flat-top power distribution along the narrow axis which is much preferred as it gives a uniform signal to noise ratio (SNR) over the image and does not have a large hot spot, allowing for a higher safe optical power of the beam and further improving the SNR.

As shown, the diverging light especially along the fast diverging axis from the fanned out rays of the Powell lens is collimated by a cylindrical lens, spherical lens, and/or an achromat lens 212, mounted to a base 110. The collimated part of the beam on the opposite axis is focused. This creates an extended beam on one axis and a collimated beam on the other axis that then produces a focused line on the retina.

The system is supported on the base 110 that is generally t-shaped with the swept laser in the bottom and the reference arm and sample arm at the top. The base is often machined out of metal such as aluminum or 3D printed plastic. The TO-can 40 is held in an L-shaped mount 114 that holds the TO-can 40 above the base.

The base 110 also has a minor well 110W for accommodating the movement of the tunable filter 52. A galvanometer cradle 110G receives the shaft of the galvanometer 50. A galvanometer clamp 115 secures the galvanometer 50 to the base 110 in the galvanometer cradle 110G.

For holding the various components, it has a series of cradles or V-groove optical element mounting locations formed into the top surface of the base. These include cats-eye focusing lens v-groove cradle 110C for holding the cat's eye focusing lens 44, collimating output lens cradle 11000 for holding the collimating output lens 60, a cats-eye collimating lens v-groove cradle 110F for holding the collimating lens 42. A line generating lens cradle 110P holds the line generating lens 210.

The light from the cylindrical collimating lens 212 passes in free space to a beam splitter 214, which is mounted on the base 110. The beamsplitter 214 divides the light between the reference arm defined by a reference arm mirror 216 and the sample arm that ends with a sample 218 such as an animal or human eye.

However, the system is equally relevant to other medical and industrial uses and can be made very portable. It can be used to image the skin to assist in diagnosing and treating a variety of skin diseases. It can be used to detect changes in skin morphology associated with conditions like skin cancer, psoriasis, and dermatitis. It can further be used to image the gastrointestinal tract, helping to detect and diagnose conditions such as Barrett's esophagus, gastric cancer, and other abnormalities. It can be used to visualize the respiratory tract, allowing for detailed imaging of airway structures and assisting in the diagnosis of conditions like asthma or chronic obstructive pulmonary disease (COPD). It can further be used to image the vocal cords, middle ear, and other structures, helping with the diagnosis and treatment of conditions affecting these areas. Many industrial applications exist in which there is relative movement between the system 100 and the structures being imaged.

The light from the sample is collected by a collection and collimating lens 220 and the light from the two arms returns to the beamsplitter 214 to be combined to form light interference in a line-field sensor 240. A lens bracket 222 mounts the collection and collimating lens 220 to the base 110.

Light enters the line-field sensor 240 through its aperture 240A to be received by the sensor chip, which is preferably CMOS or CCD device. These are silicon devices that work at the 800-900 nm wavelength. One commercially available camera is the NECTA series sold by Alkeria Srl. This CMOS-sensor device has a USB-3 interface having at least 1024 and preferably 2048 or more pixels arranged in a line. The pixel sizes range from about over 2 micrometers to as large as 10 micrometers in different CMOS and CCD sensors.

The digital output from the line-field sensor 240 is readout by the processor 300. The results can be stored in the processor and/or displayed on display. The Fourier transform of the interference light reveals the profile of scattering intensities at different path lengths, and therefore scattering as a function of depth (z-direction) in the sample (see for example Leitgeb et al, “Ultrahigh resolution Fourier domain optical coherence tomography,” Optics Express 12(10):2156 2004). The profile of scattering as a function of depth is called an axial scan (A-scan). A set of A-scans measured at neighboring locations in the sample produces a cross-sectional image (tomogram or B-scan) of the sample. A collection of B-scans makes up a data cube or cube scan as the line from the system 100 is scanned or swept over the sample 218.

Typically, an additional galvanometer driven scanning mirror is provided between the beamsplitter 214 and the sample, so that the line-shaped beam of light is scanned in one axis.

In terms of packaging, the reference arm minor 216 is held in a mirror bracket 250 that is moved by a translation stage 252 for reference arm pathlength adjustment. The translation stage 252 is mounted to the base 110 and specifically an arm of the base.

The beamsplitter 214 is also mounted to the base 110. The line-field sensor 240 is mounted to a camera bracket 254 that is mounted to the base 110.

While this invention has been particularly shown and described with references to preferred embodiments thereof, it will be understood by those skilled in the art that various changes in form and details may be made therein without departing from the scope of the invention encompassed by the appended claims.

What is claimed is:

1. An integrated line-field swept source OCT system, comprising:
a base;
a swept laser on the base;
a beamsplitter on the base for dividing the beam from the swept laser between a reference arm and a sample arm; and
a line-field sensor for detecting light from the reference arm and the sample arm.

2. The system of claim 1, further comprising a cylindrical lens mounted to the base for conditioning the light from the swept laser to the beamsplitter.

3. The system of claim 1, further comprising a bracket for mounting the line-field sensor to the base.

4. The system of claim 1, further comprising a line generating lens for forming a line from the beam from the laser.

5. The system of claim 4, wherein the line generating lens forms a less Gaussian and more of a flat-top power distribution of the light from the laser.

6. The system of claim 4, wherein the line generating lens is a Powell lens.

7. The system of claim 1, wherein the base is generally t-shaped with the swept laser in the bottom and the reference arm and sample arm at the top.

8. The system of claim 1, further comprising a translation stage for changing a length of the reference arm.

9. The system of claim 1, wherein the swept laser includes a gain chip for amplifying light in a laser cavity, a collimating lens for collimating light from the gain chip, an end reflector of the laser cavity, a focusing lens for focusing the collimated light on the end reflector, a thin film bandpass filter between the collimating lens and the focusing lens, and at least one angle control actuator for changing the angle of the thin film filter to the collimated light.

10. The tunable laser of claim 9, wherein the gain chip is a GaAlAs chip.

11. The tunable laser of claim 9, wherein the gain chip is mounted in a TO-can hermetic package.

12. The tunable laser of claim 9, wherein a pass band of the thin film bandpass filter is between 0.05 nanometers (nm) and 5 nm wide, full width at half maximum (FWHM).

13. The tunable laser of claim 9, wherein a pass band of the thin film bandpass filter is between 0.1 nm and 2 nm wide, FWHM.

14. The tunable laser of claim 9, wherein the at least one angle control actuator is a galvanometer.

15. The tunable laser of claim 9, wherein the at least one angle control actuator is a servomechanism.

16. An integrated line-field swept source OCT system, comprising:
a base;
a swept laser on the base;
a beamsplitter on the base for dividing the beam from the swept laser between a reference arm and a sample arm;
a line-field sensor for detecting light from the reference arm and the sample arm; and
a line generating lens for forming a line from the beam from the laser.

17. The system of claim 16, wherein the line generating lens forms a less Gaussian and more of a flat-top power distribution of the light from the laser.

18. The system of claim 16, wherein the line generating lens is a Powell lens.
